(19) United States
(12) Patent Application Publication
(10) Pub. No.: US 2023/0255630 A1
(43) Pub. Date: Aug. 17, 2023

(54) WOUND CLOSURE DEVICE FOR WIDE TREATMENT AREAS

(71) Applicants: Cheuk Yin Paul Leung, Bellevue, WA (US); Ronald J. Berenson, Mercer Island, WA (US)

(72) Inventors: Cheuk Yin Paul Leung, Bellevue, WA (US); Ronald J. Berenson, Mercer Island, WA (US)

(21) Appl. No.: 18/140,822

(22) Filed: Apr. 28, 2023

Related U.S. Application Data
(63) Continuation of application No. 17/249,083, filed on Feb. 19, 2021.
Application No. 17/249,083 is a continuation-in-part of application No. 17/163,756, filed on Feb. 1, 2021.
Application No. 17/163,756 is a continuation of application No. 15/446,999, filed on Mar. 1, 2017, now Pat. No. 10,939,912.
(60) Provisional application No. 62/978,454, filed on Feb. 19, 2020.

(51) Int. Cl. A61B 17/08; A61B 17/064

(52) U.S. Cl. CPC A61B 17/085; A61B 17/064; A61B 2017/00862

(57) ABSTRACT

A wound closure device includes an elastic substrate and a plurality of microstructure arrays coupled to the elastic substrate via an adhesive layer. The device is long enough or wide enough to treat large wounds with the plurality of microstructure arrays distributed along a length of the wound.

### CLAIM OF PRIORITY

The present application is a continuation-in-part of U.S. patent application Ser. No. 17/249,083 filed Feb. 19, 2021, which is a non-provisional of, and claims the benefit of U.S. Provisional Patent Application No. 62/978,454 (Attorney Docket No. 5173.008PRV) filed on Feb. 19, 2020; the entire contents of these applications are incorporated herein by reference.

The present application is a continuation-in-part of U.S. patent application Ser. No. 17/163,756 (Attorney Docket No. 5173.003US2) filed Feb. 1, 2021, which is a continuation of U.S. patent application Ser. No. 15/446,999 filed on Mar. 1, 2017, now issued as U.S. Pat. No. 10,939,912; the entire contents of these applications are incorporated herein by reference.

### BACKGROUND

Existing devices, compositions, and methods for closing and treating a wound may range from simple over-the-counter products, such as dressings, wraps, bandages, adhesive bandages, butterfly strips, and surgical tape, to more specialized products, such as sutures and staples, depending on the type and severity of the wound, the skill of the caregiver, etc. While these treatments are promising, there may be challenges associated with some, and thus improved wound treatment devices are desirable.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of a wound closure device for treating a longer wound.

FIG. 2 highlights additional features of the wound closure device in FIG. 1.

FIG. 3 shows a wound closure device with an application tab.

FIG. 4A shows a wound closure device partially packaged.

FIG. 4B shows a multi-pack of wound closure devices partially packaged.

FIG. 4C shows an exploded view of the wound closure device partially packaged in FIG. 4A.

FIGS. 5A-5B show optional features of the wound closure device of FIG. 1.

FIGS. 6A-6B show an example of a wound closure device with individual microstructures.

FIGS. 7A-7B show another example of a wound closure device with individual microstructures.

FIGS. 8A-8B show yet another example of wound closure device with individual microstructures.

FIGS. 9A-9B show still another example of a wound closure device with individual microstructures.

### DETAILED DESCRIPTION

Examples of devices and packaging are described herein and generally relate to systems, apparatus, and methods for wound closure, and in particular to a wound closure device having one or more microstructures. However, one of skill in the art will appreciate that is it not intended to be limiting and the devices and packaging described herein may be used for other purposes.

Existing devices, compositions, and methods for closing and treating a wound may range from simple over-the-counter products, such as dressings, wraps, bandages, adhesive bandages, butterfly strips, and surgical tape, to more specialized products, such as sutures and staples, depending on the type and severity of the wound, the skill of the caregiver, etc. Although sutures and staples can be quite effective at closing wounds, proper application requires a trained specialist. Additionally, the application of sutures or staples is an invasive and painful procedure that frequently requires the use of an anesthetic. Furthermore, these procedures can leave unsightly scars, both from secondary insertion holes and from varying tensions applied to the laceration or surgical incision as a result of variations in suture or staple spacing and depth. For sutures, tension also can vary depending how tightly the suture is tied. Moreover, these skin closure techniques necessitate follow-up visits to a hospital or doctor's office for removal of the sutures or staples. This can be a problem not only for scheduled removal, but an even bigger issue if infection occurs since this requires removal of the sutures or sutures to reopen and clean the wound. Additionally, simply covering the wound with a bandage, such as an adhesive bandage, a butterfly closure strip, or surgical tape, is usually not sufficient to close more severe or deeper wounds, such as dermal wounds, because the adhesives used to attach devices such as adhesive bandages, butterfly closure strips, and surgical tape are not adequate to close these wounds without detaching or creep which reduces of eliminates the force drawing the edges of the tissue around the wound into apposition. Skin moisture adds to the problem by further reducing adherence of the adhesive-based bandages to the skin, which may lead to the premature release of the bandage from the skin and wound site before closure of the wound and proper healing. Also, for larger wounds, adhesive bandages may not conform to the contours of the anatomy such as an incision around a knee or a hip, resulting in reduced adherence of the device to the wound. Adhesives used can induce symptomatic allergic and inflammatory reactions and therefore in some situations it may be desirable to include other means for attaching the wound closure device to the wound. For at least some of these reasons, improved wound closure devices are desirable. At least some of these issues will be addressed by the examples of wound closure devices described herein.

Wider or longer devices containing microstructures may be desirable for several reasons. To produce wider devices, more than one microstructure array is attached to the backing. Doing so can produce devices that are a broad range of widths from 2 times to up to 50 times, the width of a device with one microstructure. Such wider devices have several advantages. First, they may enable more rapid closure of wounds. Faster procedures save time. Since operating rooms and emergency rooms cost roughly $50 and $20 per minute, respectively, saving a few minutes can translate into savings of hundreds of dollars per procedure. Saving time also allows more procedures to be performed per day which increases throughput and thus revenue for practitioners and medical facilities. More rapid procedures are a particular advantage in surgeries where extended time in the OR is associated with more complications. Second, closure is more consistent since fewer devices are placed reducing variability when closing wounds. Third, fatigue resulting from placing many devices results in increasingly poor placement during placement of latter devices applied to close the wound. The examples of devices disclosed herein may address at least some of these challenges.

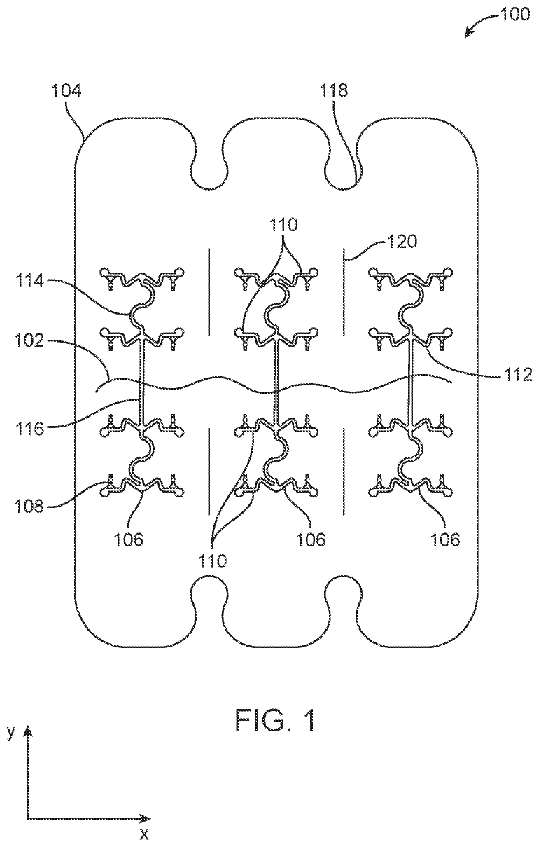

FIG. 1 shows an example of a wound closure device 100 that may be used to treat longer treatment areas than addressed by just a single narrow wound closure strip. For example, the device 100 of FIG. 1 may be used to treat incisions 102 or other wounds on a knee, a hip, abdomen, pelvis, back, chest, an extremity, or anywhere on the body. The device may be used to treat long wounds as represented in the figure by the long wavy line 102 extending horizontally across the figure. Wounds closed with the device may be linear, curvilinear, or jagged.

Some aspects of the device may take a form similar to other devices disclosed in US Patent Publication Nos. 2015/0305739 and 2017/0333039, previously incorporated by reference, except that it has been modified to treat longer wounds. Thus, the device 100 may include an upper stretchable substrate layer 104 and a lower set of one or more arrays 106 with microstructures 108 such as microstaples that attach the device to the skin or tissue adjacent the wound. Further details on the structure of the device which distinguish it from previously disclosed examples are discussed below.

In this example, the length of the device may vary depending on the length of the wound being treated and thus may be any length, and this distance will be referred to as the length of the device. The length of the device extends in the direction substantially parallel to the wound, and is represented by dimension x in FIG. 1. Length may be any desired length, for example from about 20 mm to 300 mm. The width of the device may also vary and may be any width, but often is constant (with the exception of some of the edge features described below), and extends generally orthogonally to the length of the device and transverse to or orthogonal to the wound 102. Width is indicated in FIG. 1 as dimension y. Width may be any desired width, for example from about 10 mm to about 75 mm. In the case of a short wound, the length of the device may actually be shorter than the width of the device using the dimensions as defined above; therefore, length does not always refer to the longest dimension. This device can also be used in multiples. An example is the use of two devices to cover the wound. Devices can either overlap one another or the devices may be applied immediately adjacent to one another with edges of adjacent devices touching one another or with a small gap in between edges, and this completely seals or substantially seals the wound, and the small gap separating devices may help to allow fluids to drain from the wound.

In FIG. 1, the lower set of arrays (on the skin contacting side of the upper substrate) includes three arrays 106 of microstructures 108 extending axially and generally parallel to the width (the y-direction) of the upper stretchable substrate layer 104 and therefore transverse to or orthogonal to the length of the device 100 (the x-direction) and direction of the wound 102. Each of the three arrays 106 may include two rows 110 of microstructures on either side of the wound 102 for a total of four rows 110 of microstructures 108 per array 106. The length and width of the microstructure arrays 106 may vary depending on the size of the wound being treated and the size of the wound closure device being used. Each row 110 includes two microstructures 108 such as microstaples coupled together with a sinusoidal or otherwise undulating strut 112 which can stretch outward under tension so the microstaples 108 move away from one another or the strut 112 can collapse under compression so the microstaples 108 move toward one another. Of course, any number of microstaples may be used and the size of the microstaples may also vary. The microstructure may have any configuration but generally has a small tissue piercing tip. The microstructures generally only penetrate the skin deep enough to secure the device to the skin without causing excessive irritation, pain, redness, inflammation, bleeding, etc. Additional details about the microstructures are disclosed in US Patent Publication Nos. 2015/0305739 and 2017/0333039; the entire contents of which have previously been incorporated herein by reference. The undulating strut 112 may have a short linear section coupled to the microstructure 108 followed by a valley or trough region which then may couple to the opposite undulating strut 112 which is coupled to the second microstructure in the same row. The valley portion is arcuate and therefore can expand or collapse in tension or compression, respectively. The undulating struts 112 on the opposite side of the wound 102 have a peak instead of the valley.

Additionally, rows 110 of microstaples or microstructures 108 may be connected together with a sinusoidal or undulating strut 114 that similarly can expand and collapse under tension or compression, respectively, thereby moving the rows 110 of microstaples or microstructure 108 inward toward one another or outward away from one another. The undulating strut 114 may include a peak and a valley (when FIG. 1 is rotated 90 degrees), or the strut 114 may alternatively be described as having a concave region facing in one direction (here, facing to the left), and a convex region facing in the same direction (here facing to the left), or the undulation may also be described as having a concave region furthest away from the wound facing in a first direction (here to the left), and a second concave region closest to the wound facing in a second direction opposite the first direction (here to the right). The undulating strut 113 on the opposite side of the wound 102 has the opposite concave/convex pattern compared to the strut 113 previously described. Therefore, the concavity furthest away from the wound may face to the right then the concavity closest to the wound may face toward the left. This is not intended to be limiting, and other patterns may be used.

A linear connector bridge 116 connects the rows 110 of microstaples or microstructure 108 on one side of the wound to the two rows 110 of microstaples or microstructure 108 on the opposite side of the wound, and therefore linear connector bridge 116 spans the wound 102 or incision. Because the connector bridge 116 is a straight rigid connector, it does not expand or contract, and therefore the rows 110 of microstaples immediately on either side of the wound maintain their linear position and distance from one another in the width direction (y-axis), but the rows may expand or collapse in the length direction (x-axis) due to the undulating connector. Thus, in this example, each array 106 includes two rows 110 of microstaples or microstructures 108 on either side of the wound 102, and each row 110 includes two microstaples or microstructure 108. This configuration allows the microstaples or microstructures 108 to move in the x and y directions in the plane of the substrate as needed. Additionally, in this example, the wound device 100 includes three arrays 106 of microstaples or microstructures 108, however this is not intended to be limiting and any number of arrays may be used in a wound closure device depending on how long and wide the device is. Or, in alternative examples, a multiple devices such as the device of FIG. 1 may be placed side-by-side with one another to create a longer length device to cover a long wound.

Thus, one, two, three, four, five, six, seven, eight, nine, ten, or more wound closure devices may be applied to a patient.

The microstaples or microstructures 108 may be formed of any material including metals, polymers, or other materials known in the art. The microstaples or microstructures 108 may be elongate protrusions extending from the row of microstructures or microstaples, and have a tissue piercing tip that can extend into tissue to anchor the device to the patient's skin. The depth of penetration is deep enough to ensure adequate retention strength yet not so deep that pain, inflammation, scarring, bleeding, etc. become an issue. Optionally, in any example the distance between microstaples along the length of the device is also constant between microstaples so that when the device is applied to a wound, any force transmitted from the wound closure device to the wound will be evenly distributed along the wound, at least initially unless there is expansion or contraction of the microstaples during application. However, in some circumstances, it may be desirable to have the microstaples spaced at varying distances from one another. In this example there are three arrays 106 in the device but this is not intended to be limiting, the device may have 1, 2, 3, 4, 5, 6, 7, 8, 9, 10, or more arrays depending on the size of the device and the wound to be treated. Additionally, any number of rows 110 of microstaples or microstructure 108, or any number of microstaples or microstructures 108 per row 110 may be used in order to ensure that the device does not fall away from the wound. The arrays, microstaples, microstructures or any other aspects of lower arrays may take the form of any of those disclosed in US Patent Publication Nos. 2015/0305739 and 2017/0333039, previously incorporated herein by reference.

The upper substrate may be any material such as a fabric or polymer with an adhesive layer or adhesive backing on the tissue facing side to allow the device to be secured to tissue in conjunction with the microstaples or microstructures which engage tissue. The adhesive may be applied uniformly across the entire backing or only around the perimeter of the substrate to ensure good adhesion and sealing against the wound. Other adhesive patterns may also be used and in this application the terms adhesive, adhesive layer and adhesive backing are used interchangeably. The microstaples or microstructures are applied to the tissue facing side of the adhesive layer of the upper substrate 104. In this example, the outer-most corners of the upper substrate may be rounded with a large radius in order to avoid having sharp ninety-degree corners. This helps reduce the chance that the sharp corners will catch on clothing or other objects resulting in unwanted peeling of the device away from the skin. The sides of the device parallel to the width of the device are generally linear although they may be any shape. A linear edge allows an adjacent device to appose the linear edge of an adjacent device, thereby allowing the device to cover the wound without unwanted gaps between adjacent devices. The sides of the device that extend along the length of the device (or transverse to or orthogonal to the width of the device) may have a series of cutouts 118 between the arrays 106. When a large adhesive bandage is applied to a wound having contours or that flexes (e.g. around the knuckles, the knee, etc.), the adhesive bandage may not always conform to the contours of the anatomy and the bandage may ripple, buckle or tent outward thereby creating additional stress on the adhesive and allowing the device to more easily fall off the patient. Therefore, adding the slots or cutouts 118 (sometimes also referred to as apertures) along the sides or edges of the upper substrate allows the substrate to better conform to the native anatomy reducing local stress on adhesive and thus reducing risk of local adhesive failure. The slots 118 may be any shape such as rectangular, square, oval, elliptical, circular, round, etc. and combinations thereof. Here the slots are arcuate with a narrow neck region and an enlarged head region, and this results in undulating or scalloped edges. Square or rectangular slots along the edge wound result in a castellated edge. In this example there are two slots on either side of the middle array, although there may be more or less, and this also depends on the overall length of the device. The cutouts may be used on the upper and lower edges of the device.

The upper substrate 104 may be a continuous flat planar sheet of material that covers the wound to help seal the wound and prevent infection. The upper substrate 104 may optionally include one or more slits 120 (sometimes also referred to as an aperture or a slot) extending through the substrate to improve flexibility and help the substrate conform to the contours of the wound as well as allowing an aperture through which fluid from the wound may drain while still substantially covering and protecting the wound.

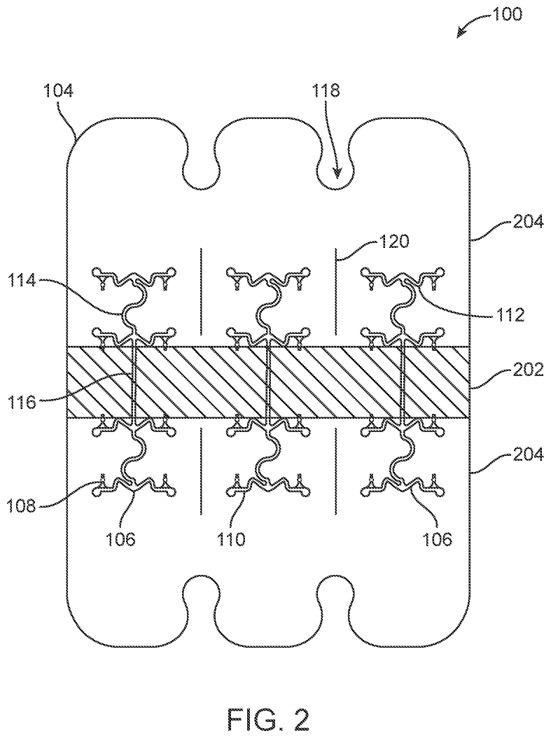

In the example of FIGS. 1-2, two linear slits 120 are disposed on either side of the wound 102 and each slit runs generally parallel to the width the device 100 (in the y-direction). The slits 120 may be positioned away from the wound so the slits are not directly disposed over the wound or they may be disposed anywhere along the upper substrate including over the wound. Additionally, the upper substrate may be also formed from a resilient material so that it may be stretched across the wound and conforms to the wound. Stretching the device across the wound applies a compressive force across the wound which may facilitate wound closure and healing since the compressive force will help appose opposite edges of tissue along the wound. The upper substrate 104 may be any size and depends on the size of the wound being treated. Other aspects of the upper substrate are described in and may be included in any of the examples disclosed herein, such as in US Patent Publication Nos. 2015/0305739 and 2017/0333039, previously incorporated herein by reference.

FIG. 2 shows an example of an assembled wound closure device such as the device 100 in FIG. 1 where the arrays have expandable and fixed regions while the upper substrate may be resilient. The device 100 may be any size depending on the size of the wound being treated. In this example, the width of the device may range from 10 mm to 75 mm and the length may range from 20 mm to 300 mm. Of course, these dimensions are not intended to be limiting, and could be any dimension, such as the length being long enough to treat a 30 mm to 500 mm long wound.

Device 100 includes cutouts 118 to allow the device to conform to the anatomy and prevent peel away as well as wound drainage slots 120. The device has three arrays 106, each with two rows 110 of microstructure 108 on each side of the wound (not shown in this figure). Therefore, each array 106 has a total of four rows 110 of microstructures. The two rows 110 of microstructures are separated from the corresponding two rows 110 of microstructure on the other side of the wound by linear bridge 116. Also, each row 110 of microstructures 108 are separated from the adjacent row by an undulating connector 114.

Microstructures 108 in a row are separated by undulating connector or bridge 112. Rounded corners on the upper substrate 104 prevent sharp edges from catching on clothing or other objects thereby reducing the chance that the device will peel away from the wound. The skin facing side of the substrate may be coated with an adhesive to help the device stick to skin. Any number of arrays of microstructures may be included to provide a longer or shorter device, therefore three arrays is not intended to be limiting.

FIG. 2 also shows the stretchable 204 and non-stretchable 202 portions of the device. The middle section of the device that spans the wound is generally non-stretchable due to the rigid connector struts or bridges 116 between the rows of microstaples on either side of the wound. So, even though the upper substrate is resilient, the rigid connector prevents or minimizes expansion or contraction in the region 202 of the device crossing the wound. This allows the compressive force of the device to be transferred to the wound so opposed tissue ends appose one another and this helps the wound close and heal. The rest of the device is generally resilient and flexible due to the resilience of the upper substrate and also due to the expandable and contractable struts that couple the microstructures together, and this helps the device conform to the anatomy to which the device is being attached. Other aspects of the device 100 in FIG. 2 are generally the same as previously described with respect to FIG. 1.

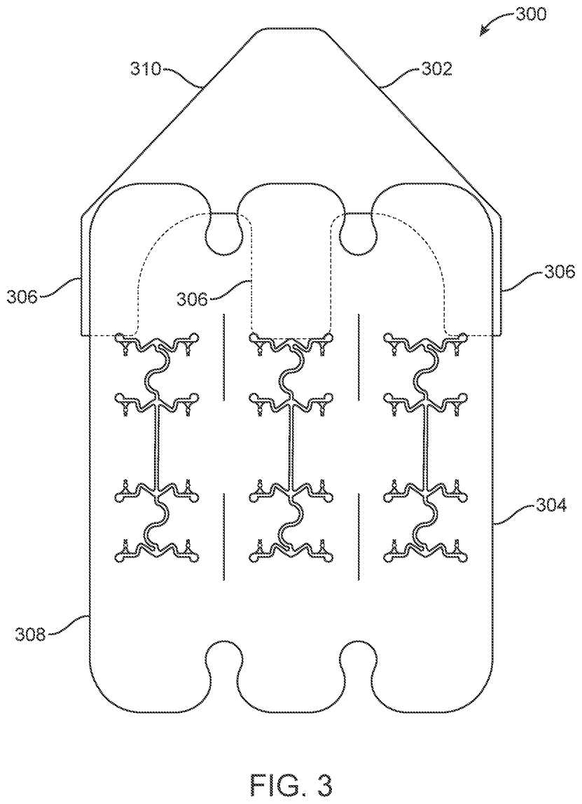

FIG. 3 illustrates an example of a wound closure application device 300 with an application tab 302 (sometimes also referred to herein as an applicator tab) releasably coupled to the wound closure device 304. The wound closure device 304 may be any of the wound closure devices disclosed herein but in this example is the same as the device illustrated in FIG. 1 and FIG. 2. The application tab 302 is releasably coupled to an edge of the device 304 along the length of the device, where the length runs in the same direction as the wound. The wound closure device 304 may extend along the length of the applicator tab 302. This helps provide support to the device 304 to help the operator keep the device 304 generally flat and planar for ease in application to the wound. Without the support of the applicator tab 302, the long flat sheet of material which makes up the device may deform under its own weight and wrinkle or deform and then the adhesive portions may stick to one another making application to the wound a challenge or rendering the device unusable. In this example, the application tab 302 has three elongate fingers (or legs) 306 which extend from the application tab 302 and are releasably coupled with the adhesive layer (not shown) on the substrate layer 308. Thus, the application tab 302 helps support the device 304 and also simultaneously minimizes surface area contact with the adhesive layer which helps the operator peel the application tab away from the adhesive layer during use with minimal force. Minimizing contact between the application tab and the adhesive layer is also advantageous since it minimizes inadvertent removal or peel away of adhesive from the substrate layer which could affect the adhesion of the device to the wound.

The application tab may also be tapered to provide a tab 310 that is easy to grasp between a thumb and finger or between fingers and also avoids having sharp corners which could catch on the operator's fingers, surgical gloves, or other adjacent objects. Thus, the example in FIG. 3 is “M” shaped where the legs 306 of the “M” are engaged with the adhesive layer and the upper portion of the “M” is tapered to provide a grip tab 310 for the operator and absent of any adhesive. A release liner (not shown in FIG. 3) which will be discussed in greater detail below will therefore be disposed over the application tab 302 such that the application tab is sandwiched between the release liner and adhesive layer of the substrate, while on other portions of the device the release liner is releasably coupled directly to the adhesive layer and a portion of the application tab is uncovered and free of the release liner. For longer devices (in the x-direction), multiple application tabs may be coupled to the device. Or, in some examples a single long application tab may be used. Optional instructions, size information, warnings or other information may be printed on the application tab 310 if desired. In use, the tab 302 may be held by an operator's hand and then the opposite end of the device is engaged with tissue on one side of the wound. The device is then pulled across the wound and a desired amount of tension or compression is applied to the device as it is applied across the wound and to the tissue on the opposite side of the wound. The tab 302 may be released from the device 304 by peeling it away, and then the device may be placed into contact with the tissue so it lays flat against the tissue and the adhesive and microstructures hold it to the tissue. The device may be applied to the wound so that the wound drainage slits are disposed on either side of the wound (e.g. above and below the wound), or in any other desired position. Other aspects of the microstructures on the device, the substrate, etc. are generally the same are previously described in FIGS. 1-2 above.

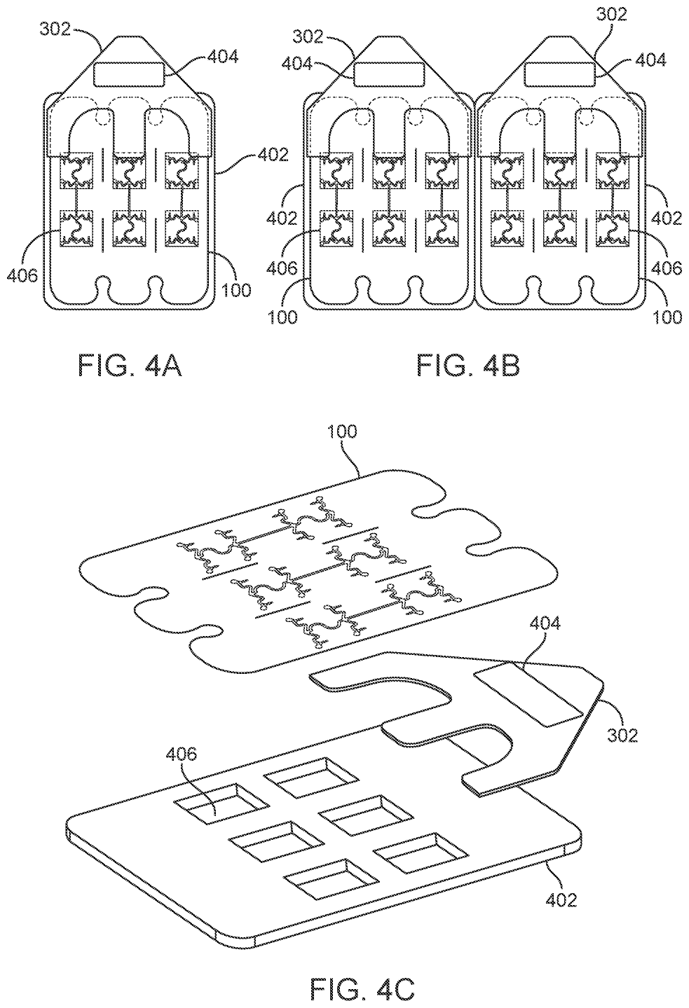

FIGS. 4A-4C illustrates examples of the wound closure device disclosed herein disposed in some of the packaging materials.

FIG. 4A shows the wound closure device 100 of FIG. 1 with the application tab 302 of FIG. 3 disposed over a release liner 402. The combined unit may then be disposed in a sterile package such as a Tyvek pouch, blister packaging, a procedure kit tray or other packaging (not illustrated) and then terminally sterilized with ethylene oxide, gamma or e-beam irradiation, plasma, or other sterilization methods known in the art. The application tab in this example includes instructions or other information 404 such as device size. The information may be disposed anywhere on the application tab, but in this example is on the tapered portion of the tab and faces upward away from the wound contacting surface. The release liner 402 includes a series of die-cut windows or apertures 406 extending through the release liner and that are sized and positioned to receive the microstaple or microstructure arrays on the wound closure device 100 so they are not damaged and the pointed tissue piercing portion of the microstructures are lifted away from the bottom of the release liner to avoid contact. The remainder of the release liner is generally a flat planar sheet of material that may be formed from a polymer and that has the same size as the wound closure device or is slightly larger. The release liner is releasably coupled to the adhesive layer on the upper substrate of the wound closure device. Some materials that may be used for the release liner can include silicone or another material that is easily peeled away from the adhesive during use, except for the area where the application tab 302 is disposed between the adhesive layer on the skin contacting surface of the upper substrate and the release liner as previously discussed above. Here, the application tab is adhesively coupled to the adhesive layer on the upper substrate and the release liner may rest against the applicator tab.

FIG. 4B shows and example where two devices 100 are packaged together. In this example, two wound closure devices such as those seen in FIG. 1 are packaged side-by-side with adjacent edges either apposed with one another or with a small gap therebetween. The devices 100 are disposed over a release liner 402 that generally takes the same form as the release liner shown in FIG. 4A above except that it is doubled to accommodate two devices. The release liner 402 may be a single integral piece, or it may be two adjacent but discrete release liners, or it may be a single piece with a perforation to allow easy separation between the two wound closure devices. The application tab in this example includes two separate application tabs 302 with one on each wound closure device 100. However, the application tab may also be one single integral tab that is coupled to both wound closure devices, or it may be two application tabs that are coupled together but has a perforation between adjacent devices 100 for easy separation. Other aspects of the device and packaging (e.g. application tab 302, information on the application tab 404, die-cut windows 406 in the release liner 402, etc.) generally take the same form as previously described above with respect to FIGS. 1-4A. In some examples, the two devices are integrally formed as a single wound closure device thereby providing an even longer device capable of treating even larger wounds. For even longer wounds, two or more devices may be applied to the wound. The devices may be individual devices applied to the wound in a side-by-side configuration, or the devices may be integrally coupled together or releasably coupled together and applied to the wound.

FIG. 4C shows and exploded view of the assembly in FIG. 4A. The wound closure device 100 may be the same as the device illustrated in FIG. 1 above, and the application tab 302 may be the same as described in FIG. 3. The release liner 402 is also the same as previously described above in FIG. 4A. FIG. 4C shows that the release liner 402 may be thicker than the either the applicator tab 404 or the wound closure device 100. This allows the wells created by aperture 406 to accommodate the thickness of the microstructures in the wound closure device 100, thereby preventing contact with or damage to the microstructures. Other aspects of FIG. 4C are the same as previously described with respect to FIGS. 4A-4B.

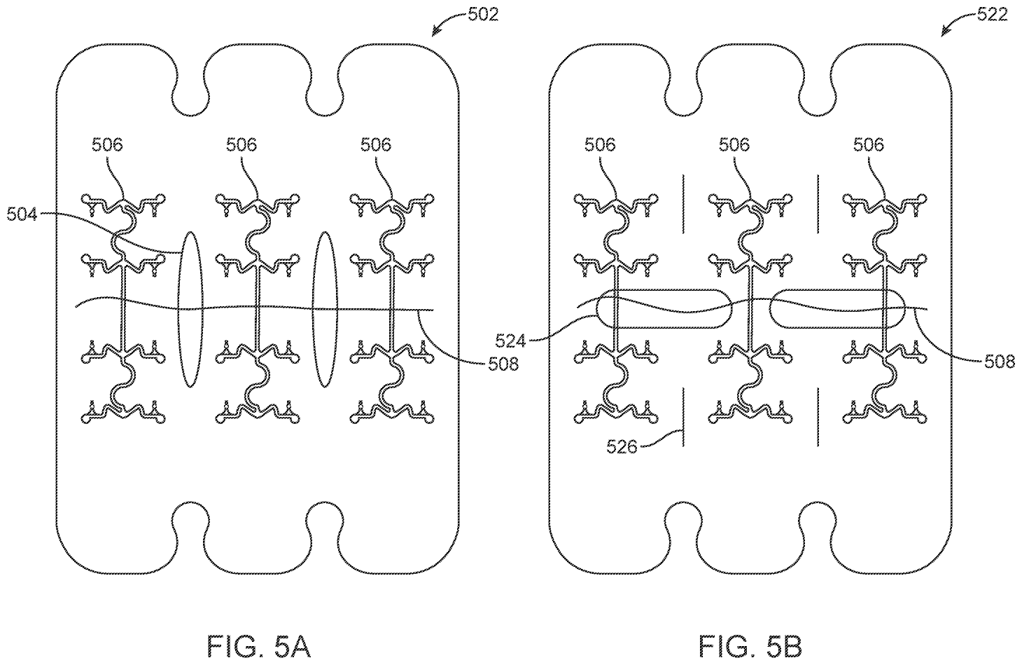

FIGS. 5A-5B illustrate examples of wound closure devices that allow fluid drainage from the wound through the wound closure device. It may be beneficial to allow fluids such as blood, serous fluid, serosanguinous fluids, or other wound related fluids to drain from the wound.

FIG. 5A shows an example of a wound closure device 502 that is substantially the same as the example in FIG. 1 with the major difference being that the optional wound drainage slits in FIG. 1 have been replaced with larger elliptical shaped slots 504 in the device for wound drainage. In this example, the elliptical slots 504 are disposed between adjacent microstructure arrays 506 and the elliptical slots 504 extend over and across the wound 508. The long axis of the elliptical slot is also substantially parallel with the width of the wound closure device 502. This example shows two elliptical slots 504, however one of skill in the art will appreciate that any number of elliptical or non-oval slots or narrow slits may be used in any position along the device. One advantage of an elliptical or other elongate shaped slots is that they still permit the slot to be disposed over the wound and allow fluid drainage from the wound even when the device has not been centered exactly over the wound. Other aspects of the wound closure device 502 are generally the same as the wound closure devices described in FIGS. 1-2 and may include the applicator tab and release backing described in FIGS. 3-4C.

FIG. 5B shows an example of a wound closure device 522 that is substantially the same as the example in FIG. 1 with the major difference being that in addition to the optional wound drainage slits in FIG. 1, larger oval shaped slots 524 have been added to the device 522. In this example, the oval shaped slots 524 are disposed between rows of microstructures 506 that are disposed on either side of the wound 508, and generally in the unstretchable portion of the device. The oval slots 524 are disposed in the upper substrate so that they will lie substantially over the wound 508 and the long axis of the oval is substantially parallel with the wound 508 and runs in the same direction as the length of the device. This example shows two ovals but one of skill in the art will appreciate that this is not limiting, and any number of oval slots may be used and they may be disposed over any portion of the upper substrate. Additionally, the wound closure device 522 also includes narrow slits 526 for wound fluid drainage and device flexibility. In this example there are two slits 526 on either side of the wound and the slits 526 are substantially parallel with the width of the wound closure device. Each slit is disposed between adjacent arrays 506 of microstructures. Other aspects of the narrow slits 526 are generally the same as previously described in FIGS. 1-3 above. Also other aspects of the wound closure device 522 are generally the same as the wound closure devices described in FIGS. 1-2 and may include the applicator tab and release backing described in FIGS. 3-4C.

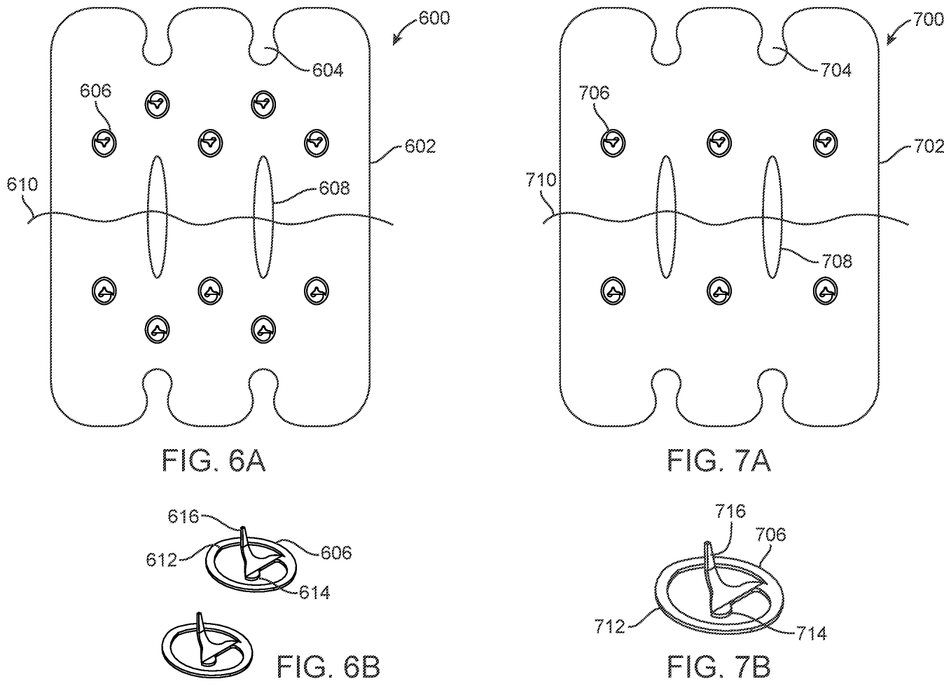

FIGS. 6A-6B show another example of a wound closure device which is similar to those previously disclosed herein but with the major difference being that instead of an array of coupled microstructures used to secure the device to the patient's skin, here the device has individual or discrete microstructures. Therefore, there still are arrays of microstructures, but the individual microstructures are not coupled to one another.

FIG. 6A shows wound closure device 600 that has an upper layer 602 that is generally the same as upper layers (also referred to as a substrate) previously described in FIGS. 1-5B above. The upper layer may be a polymer or other material that is resilient and stretchable. It is generally flat and planar and may have an adhesive layer on the skin contacting side. The corners of the upper layer 602 may be rounded in order to break the edges so they are less likely to catch on clothing or other object and peel away from the skin.

Optional slits or slots 608 may be disposed anywhere on the device to facilitate wound drainage. Here the slots 608 are elliptical in shape and are generally oriented so that the longitudinal axis of the slot 608 is transverse to the wound 610. This ensures that at least some portion of the slot will be disposed easily over the wound 610 to allow fluid drainage without requiring precise application of the device to the wound. The slots 608 or slits may be any of shapes or orientations previously described above in FIGS. 1-5B.

The wound closure device 600 may also optionally include any of the slots 604 disposed in the upper and lower edges of the device. In this example the upper and lower edges run generally the same direction as the wound. This is not intended to be limiting and the slots 604 may be disposed on any edge of the device 600. The slots 604 as previously described allow the upper layer to flex and conform to the patient where the anatomy is not flat, or where the anatomy moves, thereby helping to reduce the possibility that the device will peel away and separate from the patient. The shape of the slots 604 may be any of the shapes previously described above in FIGS. 1-5B.

Unlike previous examples where the device is attached to the skin with microstructures that are coupled to one another with struts or connectors, in this example the wound closure device is coupled to the skin of a patient with individual microstructures 606 or microstaples that are coupled to the adhesive layer on the skin contacting surface of the upper layer 602. Here, there are two rows of microstructures 606 on either side of the wound 610. Each row has at least one microstructure. In this example the row farthest away from the wound has two microstructures separated by a gap and the row closest to the wound has three microstructures separated by a gap. The rows may be staggered relative to one another or they may be in phase with one another. The microstructures or microstaples may be any of the microstructures of microstaples disclosed herein. Therefore, the array of microstructures is a plurality of unconnected, discrete, or independent microstructures.

FIG. 6B shows a close up view of a microstructure 606 from the device 600 in FIG. 6A. Here, the microstructure includes a base 612 which in this example is an annular base that can adhere to the adhesive on the skin facing surface of the upper layer 602 (best seen in FIG. 6A). The annular base has an arm 614 that extends radially inward from the perimeter of the annular base and the microstructure or microstaple 616 is coupled to the arm and extends upward and away from the arm 614 in the direction of the skin, away from the adhesive side of the upper layer. Thus, the microstructures are independent and discrete from one another and may be uncoupled to one another. The microstructures generally have a tissue piercing end that is configured to be disposed in tissue deep enough to anchor the device to the patient but without causing excessive irritation, pain, redness, inflammation, bleeding, etc. as previously disclosed above. The microstructures may take the form of any microstructure disclosed herein, or disclosed in US Patent Publication Nos. 2015/0305739, 2017/0333039, or U.S. patent application Ser. No. ______ (Attorney Docket No. 5173.005US1) filed on the same day as the present application; all previously incorporated by reference.

FIGS. 7A-7B show another example of a wound closure device 700 that is similar to the example in FIGS. 6A-6B with the major difference being the pattern of microstructures or microstaples used to secure the device to the patient's skin. Again, the microstructures are uncoupled to one another, therefore there are arrays of microstructures but the individual microstructures remain discrete and independent of one another.

In FIG. 7A, wound closure device 700 includes an upper layer 702 with slots 704 that help the device conform to the patient and wound drainage slots or slits 708. The upper layer, slots 704 and slots or slits 708 are generally the same as those previously described above with references to FIGS. 6A-6B. Here, the wound closure device 700 is secured to the patient's skin with microstructures or microstaples 706 that are also substantially the same as those in FIGS. 6A-6B but they are disposed in a difference pattern. Here, one row of microstructures is on either side of the wound 710. Each row includes three discrete, unconnected microstructures that are separated by a gap and that are generally the same as previously described in FIGS. 6A-6B.

FIG. 7B shows a close up of the microstructures 706 which as previously discussed include a base 712 which in this example is an annular base having an arm 714 coupled to the base and that extends radially inward from the perimeter of the base. The microstructure here again has a tissue piercing tip 716 which is coupled to the arm and is generally the same as the microstructures previously described above in FIGS. 6A-6B. Other aspects of the microstructures or microstaples generally may take the same form as any of those described herein.

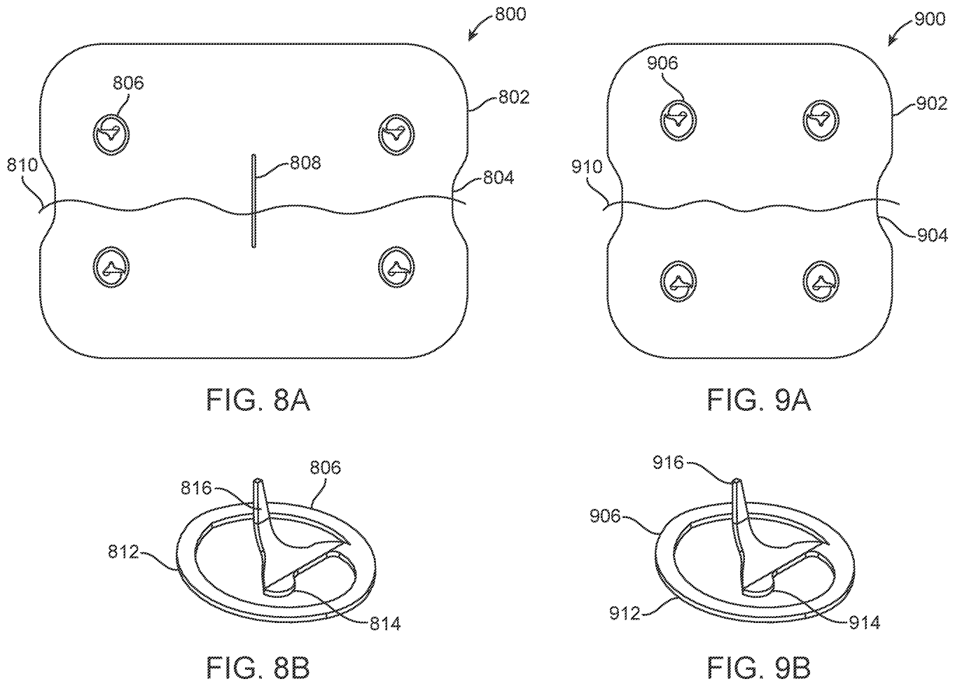

FIGS. 8A-8B show another example of a wound closure device that is similar to the examples above in FIGS. 6A-7B, but with a different microstructure pattern and a different wound drainage slit. Also, the upper layer has a slightly different shape.

FIG. 8A shows wound closure device 800 having an upper layer 802 that is generally the same as upper layers previously described in FIGS. 1-7B above. The upper layer may be a polymer or other material that is resilient and stretchable. It is generally flat and planar and may have an adhesive layer on the skin contacting side. The corners of the upper layer 802 may be rounded in order to break the edges so they are less likely to catch on clothing or other object and peel away from the skin.

Optional slits or slots 808 may be disposed anywhere on the device to facilitate wound drainage. Here the slits or slots 808 are thin and linear in shape and are generally oriented so that the longitudinal axis of the slit or slot 808 is transverse to the wound 810. This ensures that at least some portion of the slot will be disposed easily over the wound 810 to allow fluid drainage without requiring precise application of the device to the wound. The slits or slots 808 or slits may be any of shapes or orientations previously described above in FIGS. 1-7B. In this example there is only one slit disposed between the microstructures or microstaples but any number of slits or slots may be used.

The wound closure device 800 may also optionally include any of the slots 804 disposed in the lateral edges of the device, which here run generally transverse or orthogonal to the wound 810. This is not intended to be limiting and the slots 804 may be disposed on any edge of the device 800. The slots 804 as previously described allow the upper layer to flex and conform to the patient where the anatomy is not flat, or where the anatomy moves, thereby helping to prevent the device from peeling away and separating from the patient. The shape of the slots 804 may be any of the shapes previously described above in FIGS. 1-7B. Here, the slot 804 is a short arcuate cutout along the midline of the device.

Unlike previous examples where the device is coupled to the skin with microstructures that are coupled to one another with struts or connectors, in this example the wound closure device is coupled to the skin of a patient with one or more arrays of microstructures or microstaples that are not coupled together. The individual microstructures 806 or microstaples are coupled to the adhesive layer on the skin contacting surface of the upper layer 802. Here, there are two rows of microstructures 806 on either side of the wound 810, or this may also be described as a column of microstructures that span across the wound 810. Each row has at least one microstructure. In this example the row above the wound has two microstructures separated by a gap and the row below the wound has two microstructures separated by a gap. The rows here are aligned with one another so that the microstructure in one row are directly over or under the microstructure in the adjacent row. However, this is not intended to be limiting the rows may also be staggered relative to one another. The microstructures or microstaples may be any of the microstructures of microstaples disclosed herein.

FIG. 8B shows a close up view of a microstructure 806 from the device 800 in FIG. 8A. Here, the microstructure includes a base 812 which in this example is an annular base that can adhere to the adhesive on the skin facing surface of the upper layer 802 (best seen in FIG. 8A). The annular base has an arm 814 that extends radially inward from the perimeter of the annular base and the microstructure or microstaple 816 is coupled to the arm and extends upward and away from the arm 814 in the direction of the skin, away from the adhesive side of the upper layer. Thus, the microstructures are disposed in arrays but are independent and discrete from one another and may be uncoupled to one another. The microstructures generally have a tissue piercing end that is configured to be disposed in tissue deep enough to anchor the device to the patient but without causing excessive irritation, pain, redness, inflammation, bleeding, etc. as previously disclosed above. The microstructures may take the form of any microstructure disclosed herein, or disclosed in the US Patent applications incorporated herein by reference.

In FIG. 9A, wound closure device 900 includes an upper layer 902 with slots 904 that help the device conform to the patient. This example does not include wound drainage slots or slits as seen in other examples but optionally may include them. The upper layer, optional slots and slots or slits 904 are generally the same as those previously described above with references to FIGS. 6A-8B. Here, the wound closure device 900 is secured to the patient's skin with microstructures or microstaples 906 that are also substantially the same as those in FIGS. 8A-8B. Here, one row of microstructures is on either side of the wound 910, or one column is disposed across the wound 910. Each row or column includes two discrete, unconnected microstructures that are separated by a gap and that are generally the same as previously described in FIGS. 8A-8B.

FIG. 9B shows a close up of the microstructures 906 which as previously discussed includes a base 912 which in this example is an annular base having an arm 914 coupled to the base and that extends radially inward from the perimeter of the base. The microstructure here again has a tissue piercing tip 916 which is coupled to the arm and is generally the same as the microstructures previously described above in FIGS. 8A-8B. Other aspects of the microstructures of microstaples generally may take the same form as any of those described herein.

In any of the examples described in FIGS. 6A-9B, the wound closure device may optionally include the application tab or the release liner previously described above in FIGS. 4A-4C. One of skill in the art will appreciate that some modification of the release liner or application tab may be required in order to accommodate the structure of the wound closure devices in FIGS. 6A-9B.

### NOTES AND EXAMPLES

The following, non-limiting examples, detail certain aspects of the present subject matter to solve the challenges and provide the benefits discussed herein, among others.

Example 1 is a wound closure device, comprising an elastic substrate having a first axis and a second axis, the first axis configured to extend along a length of a wound and the second axis configured to extend transversely across the wound; an adhesive layer coupled to the elastic substrate; and one or more microstructure arrays each having a length along a longitudinal axis of the one or more microstructure arrays, the one or more microstructure arrays coupled to the elastic substrate via the adhesive layer, wherein the length of the one or more microstructure arrays is oriented transversely to the first axis of the elastic substrate and substantially parallel to the second axis of the substrate.

Example 2 is the device of Example 1, wherein the elastic substrate comprises four corners, and at least some of the four corners are rounded.

Example 3 is the device of any of Examples 1-2, wherein the elastic substrate comprises two side edges that extend in a direction transversely to the wound, and wherein the elastic substrate comprises top and bottom edges that extend in a substantially similar direction as the wound, the top and bottom edges having slots disposed therein.

Example 4 is the device of any of Examples 1-3, wherein the slots form a scalloped or castellated edge along the top or bottom edges, the slots configured to impart flexibility to the device so that it is configured to conform to a patient's skin.

Example 5 is the device of any of Examples 1-4, wherein the elastic substrate comprises a plurality of slots disposed therethrough, the plurality of slots configured to allow fluid drainage from the wound through the wound closure device, or wherein the plurality of slots facilitate conformance of the wound closure device to skin on a patient and flexibility of the wound closure device.

Example 6 is the device of any of Examples 1-5, wherein the plurality of slots is oriented in a direction substantially parallel with the second axis of the elastic substrate.

Example 7 is the device of any of Examples 1-6, wherein the plurality of slots is oriented in a direction substantially parallel with the first axis of the elastic substrate.

Example 8 is the device of any of Examples 1-7, wherein at least one of the one or more microstructure arrays comprise a first end and second end opposite the first end, the first end having a plurality of rows of microstaples and the second end having a plurality of rows of microstaples, the first end configured to be disposed on one side of the wound and the second end disposed on an opposite side of the wound.

Example 9 is the device of any of Examples 1-8, wherein the plurality of rows of microstaples on the one end are separated from the plurality of rows of microstaples on the second end by a rigid connector disposed across the wound.

Example 10 is the device of any of Examples 1-9, wherein a row of microstaples from the plurality of rows of microstaples comprises two microstaples separated by a flexible connector configured to expand or collapse thereby moving the two microstaples closer together or farther apart.

Example 11 is the device of any of Examples 1-10, wherein two rows of microstaples from the plurality of rows of microstaples are separated by a flexible connector configured to expand or collapse thereby moving the two rows of microstaples closer together or further apart.

Example 12 is the device of any of Examples 1-11, further comprising an applicator tab releasably coupled to the elastic substrate.

Example 13 is the device of any of Examples 1-12, wherein the applicator tab comprises a plurality of legs releasably coupled to the elastic structure and a tab configured to be grasped by an operator.

Example 14 is the device of any of Examples 1-13, further comprising a release liner releasably coupled to the elastic substrate.

Example 15 is the device of any of Examples 1-14, wherein the release liner comprises a plurality apertures therethrough, the plurality of apertures configured to receive at least a portion of the plurality of microstructure arrays.

Example 16 is the device of any of Examples 1-15, wherein the one or more microstructure arrays comprise a plurality of microstructure arrays.

Example 17 is the device of any of Examples 1-16, wherein the one or more microstructure arrays comprise a plurality of discrete unconnected microstructures.

Example 18 is a method of treating a wound, the method comprising: providing wound closure device, the wound closure device comprising an elastic substrate with an adhesive layer coupled thereto, and one or more microstructure arrays coupled to the elastic substrate via the adhesive layer; anchoring one end of the wound closure device to tissue adjacent a first side of the wound, wherein anchoring comprises attaching a plurality of arrays of microstructures to the tissue adjacent the first side of the wound; applying tension to the wound closure device and drawing opposite ends of the wound together; disposing the wound closure device across the wound; and anchoring a second end of the wound closure device opposite the first end to tissue adjacent a second side of the wound opposite the first side of the wound, wherein anchoring the second end comprises attaching a second plurality of arrays of microstructures to the tissue adjacent the second side of the wound.

Example 19 is the method of Example 18, further comprising applying the wound closure device uniformly over the wound, wherein slits, slots, or cutouts in the device allow the wound closure device to conform to contours of the wound.

Example 20 is the method of any of Examples 18-19, further comprising allowing fluids to drain from the wound via slits or an aperture disposed through the wound closure device.

Example 21 is the method of any of Examples 18-20, wherein anchoring the one end or anchoring the second end comprises engaging a microstructure or a microstaple on the wound closure device with tissue adjacent the wound.

Example 22 is the method of any of Examples 18-21, wherein applying tension to the wound closure device comprises grasping and pulling on an application tab releasably coupled to the wound closure device.

Example 23 is the method of any of Examples 18-22, further comprising removing the wound closure device from a release liner having a plurality of apertures that receive at least a portion of the plurality of microstructure arrays.

Example 24 is the method of any of Examples 18-23, wherein the one or more microstructure arrays comprise a plurality of microstructure arrays.

Example 25 is the method of any of Examples 18-24, wherein the one or more microstructure arrays comprise a plurality of discrete unconnected microstructures.

In Example 26, the apparatuses or method of any one or any combination of Examples 1-25 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A wound closure device, comprising:
an elastic substrate having a first axis and a second axis, the first axis configured to extend along a length of a wound and the second axis configured to extend transversely across the wound;
an adhesive layer coupled to the elastic substrate; and
a plurality of microstructure arrays coupled to the elastic substrate via the adhesive layer, wherein a longitudinal length of each microstructure array of the plurality of microstructure arrays is oriented transversely to the first axis of the elastic substrate and substantially parallel to the second axis of the substrate.

2. The device of claim 1, wherein the elastic substrate comprises four corners, and at least some of the four corners are rounded.

3. The device of claim 1, wherein the elastic substrate comprises two side edges that extend in a direction transversely to the wound, and wherein the elastic substrate comprises top and bottom edges that extend in a substantially similar direction as the wound, the top and bottom edges having slots disposed therein.

4. The device of claim 3, wherein the slots form a scalloped or castellated edge along the top or bottom edges, the slots configured to impart flexibility to the device so that it is configured to conform to a patient's skin.

5. The device of claim 1, wherein the elastic substrate comprises a plurality of slots disposed therethrough, the plurality of slots configured to allow fluid drainage from the wound through the wound closure device, or wherein the plurality of slots facilitate conformance of the wound closure device to skin on a patient and flexibility of the wound closure device.

6. The device of claim 5, wherein the plurality of slots are oriented in a direction substantially parallel with the second axis of the elastic substrate.

7. The device of claim 5, wherein the plurality of slots are oriented in a direction substantially parallel with the first axis of the elastic substrate.

8. The device of claim 1, wherein each microstructure array of the plurality of microstructure arrays comprise a first end and second end opposite the first end, the first end having a plurality of rows of microstaples and the second end having a plurality of rows of microstaples, the first end configured to be disposed on one side of the wound and the second end disposed on an opposite side of the wound.

9. The device of claim 8, wherein the plurality of rows of microstaples on the one end are separated from the plurality of rows of microstaples on the second end by a rigid connector disposed across the wound.

10. The device of claim 8, wherein a row of microstaples from the plurality of rows of microstaples comprises two microstaples separated by a flexible connector configured to expand or collapse thereby moving the two microstaples closer together or farther apart.

11. The device of claim 8, wherein two rows of microstaples from the plurality of rows of microstaples are separated by a flexible connector configured to expand or collapse thereby moving the two rows of microstaples closer together or further apart.

12. The device of claim 1, further comprising an applicator tab releasably coupled to the elastic substrate.

13. The device of claim 12, wherein the applicator tab comprises a plurality of legs releasably coupled to the elastic structure and a tab configured to be grasped by an operator.

14. The device of claim 1, further comprising a release liner releasably coupled to the elastic substrate.

15. The device of claim 14, wherein the release liner comprises a plurality apertures therethrough, the plurality of apertures configured to receive at least a portion of the plurality of microstructure arrays.

16. The device of claim 1, wherein the one or more microstructure arrays comprise a plurality of discrete unconnected microstructures.

17. A wound closure device, comprising:
an elastic substrate including a first axis and a second axis, the first axis configured to extend along a length of a wound and the second axis configured to extend transversely across the wound, the elastic substrate further including an adhesive layer; and
a plurality of microstructure arrays distributed along a length of the first axis and secured to the elastic substrate via the adhesive layer, wherein each microstructure array of the plurality of microstructure arrays includes at least a first microstaple configured to engage tissue on a first side of the wound and an opposing second microstaple configured to engage tissue on a second side of the wound.

18. A method of treating a wound, the method comprising:
providing a wound closure device, the wound closure device comprising an elastic substrate with an adhesive layer coupled thereto, and a plurality of microstructure arrays coupled to the elastic substrate via the adhesive layer along a length of the wound closure device extending parallel to the wound;
anchoring one end of the wound closure device to tissue adjacent a first side of the wound, wherein anchoring comprises attaching a first side of each microstructure array of the plurality of microstructure arrays to the tissue adjacent the first side of the wound;
applying tension to the wound closure device and drawing opposite ends of the wound together;
disposing the wound closure device across the wound; and
anchoring a second end of the wound closure device opposite the first end to tissue adjacent a second side of the wound opposite the first side of the wound, wherein anchoring the second end comprises attaching a second side of each microstructure array of the plurality of microstructure arrays to the tissue adjacent the second side of the wound.

19. The method of claim 18, further comprising applying the wound closure device uniformly over the wound, wherein slits, slots, or cutouts in the device allow the wound closure device to conform to contours of the wound.

20. The method of claim 18, further comprising allowing fluids to drain from the wound via slits, slots, or an aperture disposed through the wound closure device.

21. The method of claim 18, wherein applying tension to the wound closure device comprises grasping and pulling on an application tab releasably coupled to the wound closure device.
